(19) United States
(12) Patent Application Publication
(10) Pub. No.: US 2021/0054294 A1
(43) Pub. Date: Feb. 25, 2021

(54) PROCESS FOR UPGRADING A PETROLEUM PRODUCT

(71) Applicant: OXFORD UNIVERSITY INNOVATION LIMITED, Botley Oxford (GB)

(72) Inventors: Zhaoxi ZHANG, Oxford (GB); Tiancun XIAO, Oxford (GB); Peter P. EDWARDS, Oxford (GB)

(21) Appl. No.: 16/305,535

(22) PCT Filed: May 30, 2017
(86) PCT No.: PCT/GB2017/051533

§ 371 Date: Nov. 29, 2018

(30) Foreign Application Priority Data
May 31, 2016 (GB) 1609545.7

(51) Int. Cl. C10G 21/16; C10L 1/02

(52) U.S. Cl. CPC C10G 21/16; C10L 1/023; C10G 2300/104; C10G 2400/02; C10L 2270/023; C10L 2200/0423; C10G 2300/202; C10G 2300/1011; C10G 2300/1003; C10L 2290/24; C10G 2300/305; C10L 2290/542; C10L 2200/0484

(57) ABSTRACT

The present invention relates to a process for upgrading a petroleum product comprising mixing the petroleum product with an upgrading solution to provide a two-phase mixture consisting of an extract phase and a raffinate phase, wherein the upgrading solution comprises a pyrolysis oil and optionally a polar organic solvent. In other aspects, the invention provides an upgrading solution comprising a pyrolysis oil and optionally a polar organic solvent and uses thereof. Furthermore, an upgraded petroleum product and raffinate phase is provided.

### FIELD OF THE INVENTION

The present invention provides a new process for upgrading petroleum products, such as FCC gasoline/diesel. Furthermore, the present invention provides upgraded petroleum products, such as gasoline or diesel, in particular by the use of an upgrading solution in an upgrading process.

### BACKGROUND OF THE INVENTION

In the 1970s, one half of the petroleum consumed worldwide was used to produce transportation fuels, by 2030 it is expected that more than two thirds of the petroleum produced will be employed for this purpose.

The effects of air pollution caused by vehicle emissions on health and the environment are well known. Since the late 1970s, concerted effort has been made to reduce the harm caused by motor transport emissions. For example, starting in the 1970s, lead was removed from gasoline and the use of aromatics such as benzene was increased to ameliorate the loss of octane number.

However, the scientific, medical and environmental communities had long advocated the removal or reduction of aromatic hydrocarbons in gasoline. Oxygenates were subsequently seen as a way to effectively reduce the aromatic hydrocarbon content in gasoline, while maintaining octane levels (Nadim et al., Energy Policy 2001, 29, 1-5).

Widespread use of oxygenates in gasoline dates back to 1979, when methyl tert-butyl ether (MTBE) was added to gasoline to substitute tetraethyl lead (TEL) and to increase the octane rating of the fuel. In the US, the federal government introduced the reformulated gasoline (RFG) program in the most polluted cities in the United States aimed at reducing the levels of highly toxic aromatics from gasoline and increasing the oxygen content of gasoline by adding larger quantities of oxygenates. The US Congress mandated the use of a minimum 2.0% oxygenate in RFG. This required the addition of 11% (MTBE) by volume.

However, due to public concern of MTBE's effect on health and the environment, MTBE was reduced from its previous levels and replaced by other oxygenates which were less harmful to health and environment. Other oxygen-containing organic compounds used in the production of RFG include ethanol, ethyl tert-butyl ether (ETBE), tert-amyl methyl ether (TAME), and di-isopropyl ether (DIPE). Although these oxygenates are deemed to be less harmful to health and environment, they are expensive to obtain, and the production processes are highly energy intensive.

Oxygenates have proven to be an effective way of reducing the levels of harmful chemicals in fuels, maintain fuel performance (octane/cetane rating etc.) and extend the life of a barrel of oil (1.0-1.5% less crude oil needs to be refined to produce base gasoline for oxygenated gasoline). The use of oxygenated gasoline was also able to reduce the ground levels of ozone by 18% during the 1996 smog season in Los Angeles. Therefore, in order to improve the performance and fuel efficiency in the combustion engines, and to reduce air toxics and ozone in highly air-polluted areas of the world, additives such as oxygenates are still added to fuels (gasoline, diesel etc.).

Furthermore, exhaust gases from motor vehicles contribute to a large extent to air pollution through their content in NOx and SOx. This has led the governments of numerous countries to adopt new regulations which aim at a drastic reduction of sulphur emissions by imposing a very low concentration of this element in fuels. The EU presently requires that member states only allow gasoline (petrol) or diesel to be placed on the market within their territory if the sulphur content is 10 ppm or less. The new regulations make it necessary to remove sulphur compounds from gasoline and diesel almost completely.

Similarly, the EU also imposes restrictions on the amount of olefins in gasoline due to health and environmental concerns. Olefins are routinely separated by various methods including distillation which is a costly and energy-intensive process due to the similar volatilities of the various components (Song et al., Tsinghua Science and Technology, 2008, 13, 730-735).

A second approach is to use chemical reactions to directly convert the olefins and sulphur compounds into other chemical compounds. Various heterogeneous zeolitic catalysts such as SAPO-11, Hβ, HMOR, HZSM-5, HZSM-5 modified with Ga₂O₃, Co—Mo/Al₂O₃, Ni—Mo/Al₂O₃ and mesoporous zeolites have therefore been introduced into FCC hydrodesulfurization (HDS) processes (Fan, Y., et al., Fuel, 2005, 84, 435; Fan, Y. et al., Microporous and Mesoporous Materials 2007, 98, 174; Viswanadham, N. et al., Fuel, 2007, 86, 1290; Zhang, P., et al., Journal of Molecular Catalysis A: Chemical 2007, 261, 139; and Huo, Q. et al., Applied Catalysis A: General 2010, 381, 101).

In the HDS processes, olefins are selectively converted into more stable paraffin or aromatic hydrocarbons. This process is not only of high cost and highly energy-intensive, but also requires specific catalysts and complex reaction conditions.

HDS processes also reduce the content of organic sulphur compounds (OSCs). Although HDS processes show satisfactory desulfurization performance and acceptable process efficiency, they require severe operating conditions and complex procedures and, considerable amounts of elemental hydrogen as a reactant—which of course is industrially prepared from natural gas (by steam reforming) with attendant high CO₂ emissions.

There is a pressing need in the art for alternative/improved processes for adjusting the olefin, sulphur and/or oxygenate content of fuels, in particular transportation fuels. The present invention provides a safer, more sustainable and/or more efficient process for reducing olefin and/or sulphur content in petroleum products and/or increasing the level of oxygenates in petroleum products.

The resultant upgraded petroleum products are stable, and benefit from one or more of a reduced olefin content, a reduced sulphur content, an increased concentration of oxygenates compared to the starting petroleum product.

### SUMMARY OF THE INVENTION

In a first aspect, the present invention relates to a process for upgrading a petroleum product comprising mixing the petroleum product with an upgrading solution to provide a two-phase mixture consisting of an extract phase and a raffinate phase, wherein the upgrading solution comprises a pyrolysis oil and optionally a polar organic solvent.

In a second aspect, the present invention relates to a raffinate phase obtainable according to a process according to the first aspect.

In a third aspect, the present invention relates to a raffinate phase obtained by a process according to the first aspect.

In a fourth aspect, the present invention relates to a process for producing an upgraded petroleum product comprising

(i) treating a petroleum product to be upgraded with an upgrading solution,

(ii) mixing the petroleum product and upgrading solution and then allowing the mixture to form two phases consisting of a raffinate phase and an extract phase, and

(iii) separating the raffinate phase from the extract phase to yield an upgraded petroleum product;

wherein the upgrading solution comprises a pyrolysis oil and optionally a polar organic solvent.

In a fifth aspect, the present invention relates to an upgraded petroleum product obtainable according to a process according to the fourth aspect.

In a sixth aspect, the present invention relates to an upgraded petroleum product obtained by a process according to the fourth aspect.

In a seventh aspect the present invention relates to an upgrading solution for upgrading petroleum products comprising a pyrolysis oil and optionally a polar organic solvent.

In an eighth aspect the present invention relates to the use of an upgrading solution for increasing the concentration of oxygenates in a petroleum product, wherein the upgrading solution comprises a pyrolysis bio-oil and optionally a polar organic solvent.

In a ninth aspect the present invention relates to the use of an upgrading solution for increasing the octane number of a petroleum product, wherein the upgrading solution comprises a pyrolysis bio-oil and optionally a polar organic solvent.

### BRIEF DESCRIPTION OF THE DRAWINGS

FIG. 1 shows an example of the phase separation between the raffinate phase and the extract phase in the process of the present invention.

FIG. 2 shows the weight change in the FCC gasoline/raffinate phase after each extraction with an upgrading solution in an example of the invention.

FIG. 3 shows a comparison by GCMS of the raffinate phase and starting FCC gasoline in an example of the invention.

FIG. 4 shows a visual comparison of the raffinate phase (a) before and (b) after one month's storage in an example of the invention.

FIG. 5 shows a comparison by GCMS of the raffinate phase before and after one month's storage in an example of the invention.

FIG. 6 shows a boiling point analysis by TGA of the raffinate phase in an example of the invention.

### DETAILED DESCRIPTION OF THE INVENTION

### Process

In one aspect, the present invention relates to a process for upgrading a petroleum product comprising mixing the petroleum product with an upgrading solution to provide a two-phase mixture consisting of an extract phase and a raffinate phase, wherein the upgrading solution comprises a pyrolysis oil and optionally a polar organic solvent.

In another aspect, the present invention relates to a process for producing an upgraded petroleum product comprising

(i) treating a petroleum product to be upgraded with an upgrading solution,

(ii) mixing the petroleum product and upgrading solution and then allowing the mixture to form two phases consisting of a raffinate phase and an extract phase, and

(iii) separating the raffinate phase from the extract phase to yield an upgraded petroleum product;

wherein the upgrading solution comprises a pyrolysis oil and optionally a polar organic solvent.

As used herein, in each aspect of the invention, the term “upgrading” and “upgraded” used in relation to a petroleum product refers to removing or reducing the concentration of one or more unwanted substances in the petroleum product, and/or imparting or enriching the petroleum product with one or more desirable substances.

In one embodiment, the term “upgrading” and “upgraded” used in relation to a petroleum product refers to removing or reducing the concentration of one or more unwanted substances in the petroleum product. In another embodiment, the term “upgrading” and “upgraded” used in relation to a petroleum product refers to imparting or enriching the petroleum product with one or more desirable substances. Typically, upgraded/upgraded is assessed relative to the petroleum product to be upgraded, i.e. the starting petroleum product prior to being subjected to the process of the invention.

In one embodiment, the unwanted substances to be removed or reduced are selected from one or more of olefins and sulphur compounds. In another embodiment, the unwanted substances consist of olefins and sulphur compounds. In another embodiment, the unwanted substances consist of sulphur compounds. In another embodiment, the unwanted substances consist of olefins.

As used herein, the term “sulphur compounds” refers to molecules containing sulphur which are commonly found in petroleum products. In one embodiment, the sulphur compounds reduced/removed by the process of the invention comprise organic sulphur compounds (OSCs). In another embodiment, the sulphur compounds consist of organic sulphur compounds. In another embodiment, the sulphur compounds reduced/removed comprise compounds selected from thiols, thioethers, disulphides, thiophenes and benzothiophenes. In another embodiment, the sulphur compounds reduced/removed are selected from thiols, thioethers, disulphides, thiophenes and benzothiophenes.

As used herein, the term “olefins” refers to unsaturated hydrocarbons. The term “olefin” may be used interchangeably with “alkene”. In one embodiment, the olefins removed are olefins commonly found in petroleum products. In another embodiment, the olefins reduced/removed by the process of the invention are linear or branched C₂ to C₁₈ olefins. In another embodiment, the olefins reduced/removed are linear, branched or cyclic C₄ to C₁₄ olefins. In another embodiment, the olefins reduced/removed are linear, branched or cyclic C₄ to C₁₂ olefins. In another embodiment, the olefins reduced/removed are linear, branched or cyclic C₄ to C₁₀ olefins.

Examples of olefins which may be reduced/removed by the process of the invention include butene, pentene, methylbutene, hexene, methylpentene, dimethylbutene, heptene, methylhexene, dimethylpentene, octene, methylheptene, nonene, decene, undecene, dodecene, cyclobutene, cyclopentene, cyclohexene, cyclohexa-1,3-diene, methylcyclopentene, cycloheptene, methylcyclohexene, dimethylcyclopentene and cyclooctene.

In one embodiment, desirable substances which may be enriched/imparted to the petroleum product/raffinate phase include oxygenates. Oxygenates are desirable in petroleum products such as gasoline, because they increase octane rating and thus allow the reduction of cancer causing aromatic compounds. Hence, oxygenates in fuel assist with policy aims to reduce CO emissions and particulates in exhaust gases.

In another embodiment, the desirable substances enriched/imparted consist of oxygenates. As used herein the term “oxygenates” refers to hydrocarbons that contain one or more oxygen atoms. In one embodiment, the oxygenates enriched/imparted are selected from one or more of ethers, esters, ketones, carboxylic acids, aldehydes and alcohols. In another embodiment, the oxygenates are selected from one or more of ethers, esters, aldehydes, ketones and alcohols. In another embodiment, the oxygenates are selected from one or more of ethers, aldehydes, ketones and alcohols. In another embodiment, the oxygenates are selected from one or more of aldehydes, ketones and alcohols. In one embodiment, the alcohols are suitably phenols. Accordingly, in one embodiment, the oxygenates enriched/imparted are phenols.

Examples of oxygenates which may be enriched/imparted in/to the petroleum product/raffinate phase include ethanol, 1-propanol, 2-propanol, 1-butanol, 2-butanol, 2-methyl-1-propanol (isobutanol), 1-pentanol, 3-methyl-1-butanol (isopentanol), methyl levulinate, ethyl levulinate, butyl levulinate, 2 methyltetrahydrofuran (MTHF), 2-methylfuran (MF), methyl tert-butyl ether (MTBE), ethyl tert-butyl ether (ETBE), tert-amyl methyl ether (TAME), diisopropyl ether (DIPE) and 2,5-dimethylfuran.

The petroleum product and the upgrading solution may be mixed by any means known in the art. Typically, the petroleum product and the upgrading solution will be intimately mixed. For instance, the petroleum product and the upgrading solution may be added to vessels, reactors or mixers commonly used in the art and the two components may be intimately mixed. Intimate mixing may comprise vigorous agitation of the two components by a mixing means. For instance, the two components may be mixed together by stirring or by shaking.

The mixing of the two components may occur more than once. For instance, after mixing the petroleum product and the upgrading solution for the first time, the resulting two phases may be mixed again, possible numerous times. The steps of contacting and formation of two phases may be continuous. Thus, the two components may pass through a mixing means before entering a separating chamber in which the first and second phases are formed. The contacting of the two components may be performed using a propeller, counter-current flow means, an agitation means, a Scheibel® column, a KARR® column or a centrifugal extractor.

The petroleum product may be repeatedly mixed multiple times with fresh batches of upgrading solution. For instance, the petroleum product may be mixed with a first batch of an upgrading solution to provide a first raffinate phase and a first extract phase. Following separation of the raffinate phase from the extract phase the raffinate phase may be mixed with a second batch of the upgrading solution to provide a second raffinate phase and a second extract phase. This cycle may be repeated multiple times.

In one embodiment the cycle of mixing the petroleum product and its raffinate with upgrading solution is repeated between 1 and 9 times. In another embodiment, the cycle is repeated between 1 and 4 times. In another embodiment, the cycle is repeated 1, 2, 3 or 4 times. In another embodiment, the cycle is repeated 4 times.

Typically, the petroleum product and upgrading solution are mixed to the extent to allow effective extraction/enrichment of the petroleum product by the upgrading solution. In respect of two immiscible solutions, the skilled person would understood that typically these solutions are intimately mixed until an emulsion is formed which is subsequently allowed to separate into two phases.

In one embodiment, the mixing is carried out at ambient temperature and pressure. Typically, a temperature of between about 18 to 28° C., more typically between about 21 and 25° C., and a pressure of about 100 kPa. Accordingly, expense and other problems associated with high temperature or pressure conditions are avoided.

Typically, the ratio of petroleum product to upgrading solution is from about 20:1 to about 1:20. In one embodiment, the ratio of petroleum product to upgrading solution is from about 15:1 to about 1:15. In one embodiment, the ratio of petroleum product to upgrading solution is from about 15:1 to about 1:10. In one embodiment, the ratio of petroleum product to upgrading solution is from about 10:1 to about 1:10. In one embodiment, the ratio of petroleum product to upgrading solution is about 10:1 to about 1:5. In one embodiment the ratio of petroleum product to upgrading solution is about 5:1 to about 1:5. In one embodiment the ratio of petroleum product to upgrading solution is about 5:1 to about 1:1. In one embodiment the ratio of petroleum product to upgrading solution is about 5:1 to about 2:1. In one embodiment the ratio of petroleum product to upgrading solution is about 5:1.

As used herein the term “raffinate phase” refers to the phase comprising/consisting essentially of/consisting of the upgraded petroleum product.

In one embodiment, the raffinate phase will have a reduced concentration of undesirable substances compared to the petroleum product prior to mixing with the upgrading solution. In another embodiment, the raffinate phase will have an increased concentration of desirable substances compared to the petroleum product prior to mixing with the upgrading solution. In another embodiment, the raffinate phase will have a reduced concentration of undesirable substances and an increased concentration of desirable substances compared to the petroleum product prior to mixing with the upgrading solution.

In one embodiment, the raffinate phase will have a reduced concentration of sulphur compounds compared to the petroleum product prior to mixing with the upgrading solution. In another embodiment, the raffinate phase will have an increased concentration of oxygenates compared to the petroleum product prior to mixing with the upgrading solution. In another embodiment, the raffinate phase will have a reduced concentration of sulphur compounds and an increased concentration of oxygenates compared to the petroleum product prior to mixing with the upgrading solution (i.e. the starting petroleum product).

In one embodiment, the concentration of sulphur compounds in the raffinate phase is reduced by about 1% to about 80% (wt. %) relative to the concentration of sulphur compounds in the starting petroleum product. In another embodiment, the concentration of sulphur compounds in the raffinate phase is reduced by about 1% to about 50% (wt. %) relative to the concentration of sulphur compounds in the starting petroleum product. In another embodiment, the concentration of sulphur compounds in the raffinate phase is reduced by about 1% to about 30% (wt. %) relative to the concentration of sulphur compounds in the starting petroleum product. In another embodiment, the concentration of sulphur compounds in the raffinate phase is reduced by about 1% to about 20% (wt. %) relative to the concentration of sulphur compounds in the starting petroleum product.

In one embodiment, the concentration of sulphur compounds in the raffinate phase is reduced by about 5% to about 80% (wt. %) relative to the concentration of sulphur compounds in the starting petroleum product. In another embodiment, the concentration of sulphur compounds in the raffinate phase is reduced by about 5% to about 50% (wt. %) relative to the concentration of sulphur compounds in the starting petroleum product. In another embodiment, the concentration of sulphur compounds in the raffinate phase is reduced by about 5% to about 30% (wt. %) relative to the concentration of sulphur compounds in the starting petroleum product. In another embodiment, the concentration of sulphur compounds in the raffinate phase is reduced by about 5% to about 20% (wt. %) relative to the concentration of sulphur compounds in the starting petroleum product.

In one embodiment, the concentration of sulphur compounds in the raffinate phase is reduced by about 10% to about 80% (wt. %) relative to the concentration of sulphur compounds in the starting petroleum product. In another embodiment, the concentration of sulphur compounds in the raffinate phase is reduced by about 10% to about 50% (wt. %) relative to the concentration of sulphur compounds in the starting petroleum product. In another embodiment, the concentration of sulphur compounds in the raffinate phase is reduced by about 10% to about 30% (wt. %) relative to the concentration of sulphur compounds in the starting petroleum product. In another embodiment, the concentration of sulphur compounds in the raffinate phase is reduced by about 10% to about 20% (wt. %) relative to the concentration of sulphur compounds in the starting petroleum product.

In one embodiment, the raffinate phase will have a reduced concentration of olefins compared to the petroleum product prior to mixing with the upgrading solution. In another embodiment, the raffinate phase will have a reduced concentration of olefins and an increased concentration of oxygenates compared to the petroleum product prior to mixing with the upgrading solution (i.e. the starting petroleum product).

In one embodiment, the concentration of olefins in the raffinate phase is reduced by about 1% to about 80% (wt. %) relative to the concentration of olefins in the starting petroleum product. In another embodiment, the concentration of olefins in the raffinate phase is reduced by about 1% to about 50% (wt. %) relative to the concentration of olefins in the starting petroleum product. In another embodiment, the concentration of olefins in the raffinate phase is reduced by about 1% to about 30% (wt. %) relative to the concentration of olefins in the starting petroleum product. In another embodiment, the concentration of olefins in the raffinate phase is reduced by about 1% to about 20% (wt. %) relative to the concentration of olefins in the starting petroleum product. In another embodiment, the concentration of olefins in the raffinate phase is reduced by about 1% to about 10% (wt. %) relative to the concentration of olefins in the starting petroleum product.

In one embodiment, the concentration of olefins in the raffinate phase is reduced by about 5% to about 80% (wt. %) relative to the concentration of olefins in the starting petroleum product. In another embodiment, the concentration of olefins in the raffinate phase is reduced by about 5% to about 50% (wt. %) relative to the concentration of olefins in the starting petroleum product. In another embodiment, the concentration of olefins in the raffinate phase is reduced by about 5% to about 30% (wt. %) relative to the concentration of olefins in the starting petroleum product. In another embodiment, the concentration of olefins in the raffinate phase is reduced by about 5% to about 20% (wt. %) relative to the concentration of olefins in the starting petroleum product. In another embodiment, the concentration of olefins in the raffinate phase is reduced by about 5% to about 10% (wt. %) relative to the concentration of olefins in the starting petroleum product.

In one embodiment, the concentration of olefins in the raffinate phase is reduced by about 10% to about 80% (wt. %) relative to the concentration of olefins in the starting petroleum product. In another embodiment, the concentration of olefins in the raffinate phase is reduced by about 10% to about 50% (wt. %) relative to the concentration of olefins in the starting petroleum product. In another embodiment, the concentration of olefins in the raffinate phase is reduced by about 10% to about 30% (wt. %) relative to the concentration of olefins in the starting petroleum product. In another embodiment, the concentration of olefins in the raffinate phase is reduced by about 10% to about 20% (wt. %) relative to the concentration of olefins in the starting petroleum product.

In one embodiment, the raffinate phase will have a reduced concentration of sulphur compounds and olefins compared to the petroleum product prior to mixing with the upgrading solution. In another embodiment, the raffinate phase will have a reduced concentration of sulphur compounds and olefins, and an increased concentration of oxygenates compared to the petroleum product prior to mixing with the upgrading solution.

In the process of the present invention, the raffinate phase tends to be of lower density than the extract phase and thus the raffinate phase will typically be the upper phase and the extract phase will typically be the lower phase.

Typically, the process further comprises separating the raffinate phase to yield an upgraded petroleum product.

The raffinate phase may be separated by any means used in the art, and is typically separated by a physical process. Said separating typically comprises physically isolating the raffinate phase, or at least some of the raffinate phase. Thus, said separating typically comprises separating at least some of the raffinate phase from the extract phase.

As the two phases will typically already be separate in the same container due to their immiscibility, said separating may simply comprise removing (e.g. by draining or decanting) at least part of the extract phase from the container comprising the extract phase and the raffinate phase. Alternatively, the raffinate phase may be removed (e.g. by draining or decanting) from the container to leave the extract phase.

In one aspect, the present invention relates to a raffinate phase obtainable by a process as defined in any of the above embodiments.

In another aspect, the present invention relates to a raffinate phase obtained by a process as defined in any of the above embodiments.

As used herein, the term “extract phase” refers to the phase comprising the upgrading solution, for instance, the upgrading solution after it has been mixed with the petroleum product. Typically, the extract phase will comprise the majority of the upgrading solution after mixing with the petroleum product. In addition to the upgrading solution, the extract phase may comprise one or more undesirable substances extracted from the petroleum product.

In one embodiment, the extract phase will have an increased concentration of sulphur compounds compared to the upgrading solution prior to mixing with petroleum product. In another embodiment, the extract phase will have an increased concentration of olefins compared to the upgrading solution prior to mixing with petroleum product. In another embodiment, the extract phase will have an increased concentration of sulphur compounds and olefins compared to the upgrading solution prior to mixing with petroleum product.

In one embodiment, the extract phase will have a decreased concentration of oxygenates compared to the upgrading solution prior to mixing with petroleum product.

In one embodiment, the extract phase will have an increased concentration of sulphur compounds, and a decreased concentration of oxygenates compared to the upgrading solution prior to mixing with petroleum product. In another embodiment, the extract phase will have an increased concentration of olefins, and a decreased concentration of oxygenates compared to the upgrading solution prior to mixing with petroleum product. In another embodiment, the extract phase will have an increased concentration of sulphur compounds and olefins, and a decreased concentration of oxygenates compared to the upgrading solution prior to mixing with petroleum product.

In one aspect, the present invention relates to a petroleum product obtainable by a process according to the present invention. Typically, the petroleum product obtainable is an upgraded petroleum product.

In another aspect, the present invention relates to a petroleum product obtained by a process according to the present invention. Typically, the petroleum product obtained is an upgraded petroleum product.

In one embodiment, the petroleum product obtainable/obtained by the process of the invention is gasoline or diesel, typically upgraded gasoline or diesel. In another embodiment, the petroleum product obtainable/obtained is gasoline, typically upgraded gasoline.

### Petroleum Product

As used herein the term “petroleum product” refers to materials derived from crude oil, plastics or rubber compounds. For instance, petroleum products include fuel oil, diesel, kerosene (e.g. jet fuel) and gasoline (which may be interchangeably referred to as petrol), hydrocarbon products derived from cracking plastic and rubber compounds (e.g. vehicle tyres). In one embodiment, petroleum products are selected from fuel oil, diesel, kerosene (e.g. jet fuel) and gasoline (which may be interchangeably referred to as petrol). In one embodiment, the petroleum products are selected from hydrocarbon products derived from cracking plastic and rubber compounds (e.g. vehicle tyres).

In one embodiment, the petroleum product to be upgraded is selected from diesel and gasoline. In another embodiment, the petroleum product is gasoline.

In one embodiment, the petroleum product to be upgraded is gasoline obtained/obtainable from fluid catalytic cracking (FCC), thermal cracking or delayed coking of hydrocarbons. Suitably, the petroleum product to be upgraded is gasoline obtained/obtainable from fluid catalytic cracking (FCC).

FCC is a process extensively used in the oil industry whereby high molecular weight hydrocarbons are converted to lower molecular weight hydrocarbons. The process is well known in the art.

Typically, FCC is a process whereby hydrocarbons having a boiling point of greater than 340° C. are converted to hydrocarbons with a lower boiling point (less than or equal to 340° C., or typically less than or equal to 200° C.) by a catalytic process. A variety of catalysts may be used in an FCC process. Typically, a solid acid catalyst such as a zeolite catalyst is used. An FCC process may convert long chain alkanes (for instance, C11-20 alkanes) to shorter chain hydrocarbons including alkanes, cycloalkanes and alkenes (for instance C3-10 alkanes, C3-10 alkenes, C4-10 cycloalkanes). Gasoline produced by an FCC process typically has a greater alkene content than gasoline obtained by fractional distillation of crude oil. Furthermore, gasoline obtainable by an FCC process may comprise organosulfur compounds in an amount of greater than or equal to 0.05 wt %. Gasoline obtained via an FCC process is commonly known as FCC gasoline.

The depletion of the conventional light oil reserves makes FCC fuel products an important crude source of transportation fuel. However, in FCC gasoline/diesel and other partially refined fuels, the levels of olefins and organosulfur compounds (OSCs) generally need to be reduced to meet increasingly stringent fuel specifications and environmental regulations.

In one embodiment, the petroleum product to be upgraded is FCC gasoline.

In one embodiment, the petroleum product to be upgraded is FCC gasoline comprising 0.1 wt % or greater organosulphur compounds. In another embodiment, the petroleum product to be upgraded is FCC gasoline comprising 0.095 wt % or greater organosulphur compounds. In another embodiment, the petroleum product to be upgraded is FCC gasoline comprising 0.09 wt % or greater organosulphur compounds. In another embodiment, the petroleum product to be upgraded is FCC gasoline comprising 0.085 wt % or greater organosulphur compounds. In another embodiment, the petroleum product to be upgraded is FCC gasoline comprising 0.08 wt % or greater organosulphur compounds.

In another embodiment, the petroleum product is FCC gasoline comprising 30 wt. % or greater of olefins. In another embodiment, the petroleum product is FCC gasoline comprising 28 wt. % or greater of olefins. In another embodiment, the petroleum product is FCC gasoline comprising 26 wt. % or greater of olefins.

### Upgrading Solution

In one aspect, the present invention relates to an upgrading solution for upgrading petroleum products comprising pyrolysis oil and optionally a polar organic solvent.

In other aspects, the present invention relates to processes which utilise an upgrading solution.

As used herein, an upgrading solution refers to a solution/liquid mixture capable of reducing/removing one or more undesirable substances from a petroleum product and/or imparting/enriching one or more desirable substances to/in a petroleum product.

In one embodiment, the upgrading solution is capable of removing or reducing the concentration of undesirable substances in the petroleum compound, wherein the undesirable substances are selected from sulphur compounds and olefins.

In one embodiment, the upgrading solution is capable of imparting or enriching the petroleum compound in oxygenates.

In one embodiment, the upgrading solution is capable of imparting or enriching the petroleum compound in oxygenates and capable of removing or reducing the concentration of undesirable substances in the petroleum compound, wherein the undesirable substances are selected from sulphur compounds and olefins.

In one embodiment, the upgrading solution is capable of imparting or enriching the petroleum compound in oxygenates and capable of removing or reducing the concentration of olefins in the petroleum compound.

In one embodiment, the upgrading solution is capable of imparting or enriching the petroleum compound in oxygenates and capable of removing or reducing the concentration of sulphur compounds in the petroleum compound.

In one embodiment, the upgrading solution is capable of imparting or enriching the petroleum compound in oxygenates and capable of removing or reducing the concentration of sulphur compounds and olefins in the petroleum compound.

In one embodiment, the upgrading solution comprises/essentially consists of/consists of a pyrolysis oil.

Pyrolysis oil is a substance known to the skilled person. Pyrolysis oil may be obtained from a number of sources including fossil fuels, plastic, rubber and biomass. In one embodiment, the pyrolysis oil is obtainable or obtained by pyrolysis of biomass, fossil fuels, plastic and/or rubber. In another embodiment, the pyrolysis oil is obtainable or obtained by pyrolysis of biomass, fossil fuels, and/or plastic. In another embodiment, the pyrolysis oil is obtainable or obtained by pyrolysis of biomass, rubber, and/or plastic. In another embodiment, the pyrolysis oil is obtainable or obtained by pyrolysis of biomass, fossil fuels, and/or rubber.

In one embodiment the pyrolysis oil is obtainable or obtained by pyrolysis of biomass, also known as pyrolysis bio-oil. Pyrolysis bio-oil is a liquid mixture comprising in addition to hydrocarbon-based products, water and various oxygenates. Suitable categories of biomass include virgin wood (i.e. from forestry or wood processing), crops (e.g. food crops, such as wheat straw and rice straw), agricultural residues (e.g. animal waste), domestic and industrial waste (e.g. food waste).

Typically, pyrolysis is carried out at high temperature (greater than 400° C.) and with very high heating rates in the absence of oxygen.

In one embodiment, the upgrading solution comprises/essentially consists of/consists of a rubber pyrolysis oil (e.g. pyrolysis oil obtainable/obtained from pyrolysis of tyres). In one embodiment, the upgrading solution comprises/essentially consists of/consists of a plastic pyrolysis oil. In one embodiment, the upgrading solution comprises/essentially consists of/consists of a fossil fuel pyrolysis oil. In one embodiment, the upgrading solution comprises/essentially consists of/consists of a pyrolysis bio-oil.

In addition to pyrolysis oil, the upgrading solution may also comprise a polar organic solvent.

In one embodiment, the upgrading solution comprises/essentially consists of/consists of a pyrolysis oil and a polar organic solvent.

As used herein, the term polar organic solvent refers to refers to an organic compound typically having a dielectric constant (ε) of greater than or equal to 10 at about room temperature (21° C.). In some instances, polar organic solvents may be considered to be those organic solvents have a dielectric constant (ε) of greater than or equal to 15. For instance, acetone has a dielectric constant (ε) of 20.7 and methanol has a dielectric constant (ε) of 32.7. Tables of dielectric constants are readily available.

In one embodiment, the polar organic solvent is an alcohol. In one embodiment, the polar organic solvent is an alcohol selected from methanol, ethanol and n-propanol, i-propanol, n-butanol, s-butanol, i-butanol and t-butanol, pentanol, methyl glycol, glycerol, ethane-1,2-diol (ethylene glycol), propane-1,2-diol (propylene glycol) and sorbitol.

In another embodiment, the polar organic solvent is selected from methanol, ethanol and n-propanol, i-propanol, methyl glycol, glycerol, ethane-1,2-diol (ethylene glycol) and propane-1,2-diol (propylene glycol).

In another embodiment, the polar organic solvent is selected from methanol, ethanol and n-propanol, i-propanol, ethane-1,2-diol (ethylene glycol) and propane-1,2-diol (propylene glycol).

In another embodiment, the polar organic solvent comprises/essentially consists of/consists of methanol or ethanol. In another embodiment, the polar organic solvent comprises/essentially consists of consists of methanol.

In another embodiment, the polar organic solvent is a carboxylic acid. Examples of carboxylic acids which the upgrading solution may comprise include methanoic acid (formic acid), ethanoic acid (acetic acid), propanoic acid, butanoic acid and pentanoic acid.

The upgrading solution may comprise further solvents such as water, an alcohol, an aldehyde, a ketone, an ether, a carboxylic acid, an ester, a carbonate, an acid anhydride, an amide, an amine, a heterocyclic compound, an imine, an imide, a nitrile, a nitro compound, a sulfoxide, and a haloalkane.

In another embodiment, the upgrading solution may further comprise a solvent selected from water, an alcohol, a ketone, an ether, an ester, an amine, a heterocyclic compound, a nitrile, a sulfoxide and a haloalkane.

In another embodiment, the upgrading solution may further a further solvent selected from water, an alcohol, a ketone, an ether, an ester, and a nitrile.

The alcohol which the upgrading solution may further comprise may be any C1-10 alcohol, typically a C1-4 alcohol. An alcohol may have the structure alkyl-OH, HO-alkylene-OH, alkenyl-OH, OH-alkenylene-OH, cycloalkyl-OH, or OH-cycloalkylene-OH.

The alcohol may be an alcohol of formula ROH or HOR′OH, wherein R is a group selected from unsubstituted or substituted C1-10 alkyl, unsubstituted or substituted C3-10 alkenyl, unsubstituted or substituted C3-10 alkynyl, unsubstituted or substituted C4-10 cycloalkyl, and unsubstituted or substituted C6-10 aryl, and R′ is a group selected from unsubstituted or substituted C2-10 alkylene, unsubstituted or substituted C2-10 alkenylene, unsubstituted or substitutued C2-10 alkynylene, unsubstituted or substituted C5-10 cycloalkylene, and unsubstituted or substituted C6-10 arylene. Typically, R and R′ are unsubstituted.

Examples of alcohols which the upgrading solution may comprise include: monohydric alcohols such as methanol, ethanol, propanol, isopropanol (propan-2-ol), butanol (butan-1-ol), s-butanol (butan-2-ol), i-butanol (2-methylpropan-1-ol), t-butanol (2-methylpropan-2-ol), cyclopentanol, pentanol, cyclohexanol, hexanol, heptanol and octanol; and polyhydric alcohols such as ethane-1,2-diol (ethylene glycol), propane-1,2-diol (propylene glycol), propane-1,3-diol, propane-1,2,3-triol (glycerol), isopropanediol, butanediol, isobutanediol, tertbutanediol, butanetriol, pentanediol, methylbutanediol, hexanediol, hexanetriol. For compounds wherein the positions of hydroxy groups are not specified, alcohols having each of the possible positions are covered. Thus, butanediol includes butane-1,2-diol, butane-1,3-diol, butane-1,4-diol and butane-2,3-diol. Ethane-1,2-diol (ethylene glycol), propane-1,2-diol (propylene glycol), propane-1,3-diol, isopropanediol, and butanediol are examples of dihydric alcohols.

The aldehyde which the upgrading solution may further comprise may be any C1-10 aldehyde, typically a C3-6 aldehyde. An aldehyde typically has the structure alkyl-CHO. Examples of aldehydes which the upgrading solution may comprise include methanal (formaldehyde), ethanal (acetaldehyde), propanal, butanal, pentanal and hexanal.

The ketone which the upgrading solution may further comprise may be any C3-10 ketone. A ketone typically has the structure alkyl-C(O)-alkyl, cycloalkyl-C(O)-alkyl, or aryl-C(O)-alkyl. The ketone may be linear, branched, or cyclic. Examples of ketones which the upgrading solution may comprise include propanone (acetone), butanone, pentan-2-one, pentan-3-one, ethyl isopropyl ketone, hexan-2-one, and hexan-3-one.

The ether which the upgrading solution may further comprise may be any C2-10 ether, i.e. an ether containing from 2 to 10 carbon atoms. An ether typically has the structure alkyl-O-alkyl or that of an alicyclic ether. The ether may be linear, branched or cyclic. Examples of ethers which the upgrading solution may further comprise include diethyl ether, ethyl isopropyl ether, dipropyl ether, diisopropyl ether and tetrahydrofuran.

The carboxylic acid which the upgrading solution may further comprise may be any C1-8 carboxylic acid. A carboxylic acid typically has the structure alkyl-COOH. The carboxylic acid may be linear, branched or cyclic. Examples of carboxylic acids which the upgrading solution may comprise include methanoic acid (formic acid), ethanoic acid (acetic acid), propanoic acid, butanoic acid and pentanoic acid.

The ester which the upgrading solution may further comprise may be any C2-10 ester. For instance, the ester may be a C1-5 alkyl C1-5 carboxylate. An ester typically has the structure alkyl-COO-alkyl. Examples of the ester which the upgrading solution may comprise include methyl formate, ethyl formate, methyl acetate, ethyl acetate, vinyl acetate, propyl acetate, isopropyl acetate, butyl acetate, isobutyl acetate, tertbutyl acetate, pentyl acetate, methyl propanoate, ethyl propanoate, propyl propanoate, and ethyl isopropanoate.

The carbonate which the upgrading solution may further comprise may be any C3-10 carbonate. A carbonate typically has the structure alkyl-OC(O)O-alkyl. Examples of the carbonate that the upgrading solution may comprise include dimethylcarbonate, ethylmethylcarbonate and diethyl carbonate. The carbonate may be propylene carbonate or trimethylene carbonate. The acid anhydride which the upgrading solution may comprise may be any C4-8 acid anhydride. An example of the acid anhydride which the upgrading solution may comprise is acetic anhydride.

The amide which the upgrading solution may further comprise be any C2-10 amide. An amide typically has the structure alkyl-CONH₂, alkyl-CONH(alkyl) or alkyl-CON(alkyl)₂.

Examples of the amide which the upgrading solution may further comprise include formamide, N-methyl formamide, dimethyl formamide, dimethyl acetamide, N-vinylacetamide, pyrrolidone, N-methyl pyrrolidone, and N-vinyl pyrrolidone.

The amine which the upgrading solution may further comprise may be any C2-15 amine. An amine typically has the structure RNH₂, R₂NH, R₃N, and H₂NR′NH₂ where R may be selected from C2-10 alkyl, C2-10 alkenyl, C2-12 alkynyl, C6-10 aryl, and C6-12 arylalkyl, and R′ may be selected from C2-10 alkylene, C2-10 alkenylene, C2-10 alkynylene, C5-10 cycloalkylene, and C6-10 arylene. The amine may be a primary, secondary or tertiary amine. The amine may comprise one or more, or two or more amine groups. The amine may be selected from mono-C2-15-alkylamines, di-C1-7-alkylamines and tri-C1-5-alkylamines. The amine may be a C2-10-alkylenediamine. Examples of the amine which the upgrading solution may comprise include ethylamine, triethylamine, tripropylamine, tributylamine, ethylenediamine, propylenediamine, diethylenetriamine, morpholine, piperidine, and quinoline.

The heterocyclic compound which the upgrading solution may further comprise may be any C3-10 heterocyclic compound. The heterocyclic compound may be any compound having from 3 to 10 carbon atoms and comprising a ring, which ring comprises a heteroatom selected from N, P, O and S. The upgrading solution may comprise a heterocyclic compound selected from furan, tetrahydrofuran, thiophene, pyrrole, pyrroline, pyrrolidine, dioxolane, oxazole, thiazole, imidazole, imidazoline, imidazolidine, pyrazole, pyrazoline, pyrazolidine, izoxazole, isothiazole, oxadiazole, pyran, pyridine, piperidine, pyridazine, and piperazine. For instance, the upgrading solution may further comprise pyridine, furan or tetrahydrofuran.

The imine which the upgrading solution may further comprise may be a C4-10 imine. The imide which the upgrading solution may further comprise may be a C4-10 imide.

The nitrile which the upgrading solution may further comprise may be a C2-10 nitrile. For instance, the upgrading solution may comprise acetonitrile or propionitrile.

The nitro compound which the upgrading solution may further comprise may be a C1-10 nitro compound. For instance, the upgrading solution may comprise nitromethane, nitroethane, nitropropane or nitrobenzene.

The sulfoxide compound which the upgrading solution may further comprise may be a C2-10 sulfoxide compound. For instance, the upgrading solution may comprise dimethylsulfoxide (DMSO). The upgrading solution may further comprise diethylsulfoxide or methylethylsulfoxide.

The haloalkane which the upgrading solution may further comprise may be any C1-10 haloalkane. For instance, the upgrading solution may further comprise dichloromethane (DCM), trichloromethane, tetrachloromethane or dichloroethane.

In a particular embodiment, the upgrading solution may further comprise a polar organic solvent selected from methanol, ethanol, propanol, isopropanol, butanol, sec-butanol, iso-butanol and at least one further solvent selected from water, ethylene glycol, propylene glycol, and propane-1,3-diol.

In another embodiment, the upgrading solution may further comprise a polar organic solvent selected from methanol, ethanol, propanol, isopropanol and at least one further solvent selected from water, ethylene glycol, and propylene glycol.

In one embodiment, the upgrading solution comprises (i) a pyrolysis oil, (ii) a polar organic solvent selected from methanol, ethanol, propanol, isopropanol, butanol, sec-butanol, iso-butanol, (iii) at least one further alcohol solvent selected from ethylene glycol, propylene glycol, and propane-1,3-diol; and (iv) water.

In one embodiment, the upgrading solution essentially consists of (i) a pyrolysis oil, (ii) a polar organic solvent selected from methanol, ethanol, propanol, isopropanol, butanol, sec-butanol, iso-butanol, (iii) at least one further alcohol solvent selected from ethylene glycol, propylene glycol, and propane-1,3-diol; and (iv) water.

In one embodiment, the upgrading solution consists of (i) a pyrolysis oil, (ii) a polar organic solvent selected from methanol, ethanol, propanol, isopropanol, butanol, sec-butanol, iso-butanol, (iii) at least one further alcohol solvent selected from ethylene glycol, propylene glycol, and propane-1,3-diol; and (iv) water.

In one embodiment, the upgrading solution comprises (i) a pyrolysis oil, (ii) a polar organic solvent selected from methanol, ethanol, propanol, isopropanol, butanol, sec-butanol, iso-butanol, (iii) ethylene glycol; and (iv) water.

In one embodiment, the upgrading solution essentially consists of (i) a pyrolysis oil, (ii) a polar organic solvent selected from methanol, ethanol, propanol, isopropanol, butanol, sec-butanol, iso-butanol, (iii) ethylene glycol; and (iv) water.

In one embodiment, the upgrading solution consists of (i) a pyrolysis oil, (ii) a polar organic solvent selected from methanol, ethanol, propanol, isopropanol, butanol, sec-butanol, iso-butanol, (iii) ethylene glycol; and (iv) water.

In one embodiment, the upgrading solution comprises (i) a pyrolysis oil, (ii) a polar organic solvent selected from methanol, ethanol, propanol, isopropanol, butanol, sec-butanol, iso-butanol, and (iii) water.

In one embodiment, the upgrading solution essentially consists of (i) a pyrolysis oil, (ii) a polar organic solvent selected from methanol, ethanol, propanol, isopropanol, butanol, sec-butanol, iso-butanol, and (iii) water.

In one embodiment, the upgrading solution consists of (i) a pyrolysis oil, (ii) a polar organic solvent selected from methanol, ethanol, propanol, isopropanol, butanol, sec-butanol, iso-butanol, and (iii) water.

In one embodiment, the upgrading solution comprises (i) a pyrolysis oil, and (ii) a polar organic solvent selected from methanol, ethanol, propanol, isopropanol, butanol, sec-butanol, iso-butanol, preferably methanol.

In one embodiment, the upgrading solution essentially consists of (i) a pyrolysis oil, and (ii) a polar organic solvent selected from methanol, ethanol, propanol, isopropanol, butanol, sec-butanol, iso-butanol, preferably methanol.

In one embodiment, the upgrading solution consists of (i) a pyrolysis oil, and (ii) a polar organic solvent selected from methanol, ethanol, propanol, isopropanol, butanol, sec-butanol, iso-butanol, preferably methanol.

In one embodiment, the upgrading solution comprises (i) a pyrolysis bio-oil, (ii) a polar organic solvent selected from methanol, ethanol, propanol, isopropanol, butanol, sec-butanol, iso-butanol, (iii) at least one further alcohol solvent selected from ethylene glycol, propylene glycol, and propane-1,3-diol; and (iv) water.

In one embodiment, the upgrading solution essentially consists of (i) a pyrolysis bio-oil, (ii) a polar organic solvent selected from methanol, ethanol, propanol, isopropanol, butanol, sec-butanol, iso-butanol, (iii) at least one further alcohol solvent selected from ethylene glycol, propylene glycol, and propane-1,3-diol; and (iv) water.

In one embodiment, the upgrading solution consists of (i) a pyrolysis bio-oil, (ii) a polar organic solvent selected from methanol, ethanol, propanol, isopropanol, butanol, sec-butanol, iso-butanol, (iii) at least one further alcohol solvent selected from ethylene glycol, propylene glycol, and propane-1,3-diol; and (iv) water.

In one embodiment, the upgrading solution comprises (i) a pyrolysis bio-oil, (ii) a polar organic solvent selected from methanol, ethanol, propanol, isopropanol, butanol, sec-butanol, iso-butanol, (iii) ethylene glycol; and (iv) water.

In one embodiment, the upgrading solution essentially consists of (i) a pyrolysis bio-oil, (ii) a polar organic solvent selected from methanol, ethanol, propanol, isopropanol, butanol, sec-butanol, iso-butanol, (iii) ethylene glycol; and (iv) water.

In one embodiment, the upgrading solution consists of (i) a pyrolysis bio-oil, (ii) a polar organic solvent selected from methanol, ethanol, propanol, isopropanol, butanol, sec-butanol, iso-butanol, (iii) ethylene glycol; and (iv) water.

In one embodiment, the upgrading solution comprises (i) a pyrolysis bio-oil, (ii) a polar organic solvent selected from methanol, ethanol, propanol, isopropanol, butanol, sec-butanol, iso-butanol, and (iii) water.

In one embodiment, the upgrading solution essentially consists of (i) a pyrolysis bio-oil, (ii) a polar organic solvent selected from methanol, ethanol, propanol, isopropanol, butanol, sec-butanol, iso-butanol, and (iii) water.

In one embodiment, the upgrading solution consists of (i) a pyrolysis bio-oil, (ii) a polar organic solvent selected from methanol, ethanol, propanol, isopropanol, butanol, sec-butanol, iso-butanol, and (iii) water.

In one embodiment, the upgrading solution comprises (i) a pyrolysis bio-oil, (ii) a polar organic solvent selected from methanol and ethanol and (iii) water.

In one embodiment, the upgrading solution essentially consists of (i) a pyrolysis bio-oil, (ii) a polar organic solvent selected from methanol and ethanol, and (iii) water.

In one embodiment, the upgrading solution consists of (i) a pyrolysis bio-oil, (ii) a polar organic solvent selected from methanol and ethanol, and (iii) water.

In one embodiment, the upgrading solution comprises (i) a pyrolysis bio-oil, and (ii) methanol.

In one embodiment, the upgrading solution essentially consists of (i) a pyrolysis bio-oil, and (ii) methanol.

In one embodiment, the upgrading solution consists of (i) a pyrolysis bio-oil, and (ii) methanol.

In one embodiment, the upgrading solution comprises pyrolysis oil in an amount of greater than or equal to about 1 wt. %. In another embodiment, the upgrading solution comprises pyrolysis oil in an amount of greater than or equal to about 5 wt. %. In another embodiment, the upgrading solution comprises pyrolysis oil in an amount of greater than or equal to about 10 wt. %.

In another embodiment, the upgrading solution comprises pyrolysis oil in an amount of about 1 wt. % to about 90 wt. %. In another embodiment, the upgrading solution comprises pyrolysis oil in an amount of about 1 wt. % to about 80 wt. %. In another embodiment, the upgrading solution comprises pyrolysis oil in an amount of about 1 wt. % to about 70 wt. %. In another embodiment, the upgrading solution comprises pyrolysis oil in an amount of about 1 wt. % to about 60 wt. %. In another embodiment, the upgrading solution comprises pyrolysis oil in an amount of about 1 wt. % to about 50 wt. %. In another embodiment, the upgrading solution comprises pyrolysis oil in an amount of about 1 wt. % to about 40 wt. %. In another embodiment, the upgrading solution comprises pyrolysis oil in an amount of about 1 wt. % to about 30 wt. %. In another embodiment, the upgrading solution comprises pyrolysis oil in an amount of about 1 wt. % to about 20 wt. %.

In another embodiment, the upgrading solution comprises pyrolysis oil in an amount of about 2 wt. % to about 90 wt. %. In another embodiment, the upgrading solution comprises pyrolysis oil in an amount of about 2 wt. % to about 80 wt. %. In another embodiment, the upgrading solution comprises pyrolysis oil in an amount of about 2 wt. % to about 70 wt. %. In another embodiment, the upgrading solution comprises pyrolysis oil in an amount of about 2 wt. % to about 60 wt. %. In another embodiment, the upgrading solution comprises pyrolysis oil in an amount of about 2 wt. % to about 50 wt. %. In another embodiment, the upgrading solution comprises pyrolysis oil in an amount of about 2 wt. % to about 40 wt. %. In another embodiment, the upgrading solution comprises pyrolysis oil in an amount of about 2 wt. % to about 30 wt. %. In another embodiment, the upgrading solution comprises pyrolysis oil in an amount of about 2 wt. % to about 20 wt. %.

In another embodiment, the upgrading solution comprises pyrolysis oil in an amount of about 5 wt. % to about 90 wt. %. In another embodiment, the upgrading solution comprises pyrolysis oil in an amount of about 5 wt. % to about 80 wt. %. In another embodiment, the upgrading solution comprises pyrolysis oil in an amount of about 5 wt. % to about 70 wt. %. In another embodiment, the upgrading solution comprises pyrolysis oil in an amount of about 5 wt. % to about 60 wt. %. In another embodiment, the upgrading solution comprises pyrolysis oil in an amount of about 5 wt. % to about 50 wt. %. In another embodiment, the upgrading solution comprises pyrolysis oil in an amount of about 5 wt. % to about 40 wt. %. In another embodiment, the upgrading solution comprises pyrolysis oil in an amount of about 5 wt. % to about 30 wt. %. In another embodiment, the upgrading solution comprises pyrolysis oil in an amount of about 5 wt. % to about 20 wt. %.

In another embodiment, the upgrading solution comprises pyrolysis oil in an amount of about 10 wt. % to about 90 wt. %. In another embodiment, the upgrading solution comprises pyrolysis oil in an amount of about 10 wt. % to about 80 wt. %. In another embodiment, the upgrading solution comprises pyrolysis oil in an amount of about 10 wt. % to about 70 wt. %. In another embodiment, the upgrading solution comprises pyrolysis oil in an amount of about 10 wt. % to about 60 wt. %. In another embodiment, the upgrading solution comprises pyrolysis oil in an amount of about 10 wt. % to about 50 wt. %. In another embodiment, the upgrading solution comprises pyrolysis oil in an amount of about 10 wt. % to about 40 wt. %. In another embodiment, the upgrading solution comprises pyrolysis oil in an amount of about 10 wt. % to about 30 wt. %. In another embodiment, the upgrading solution comprises pyrolysis oil in an amount of about 10 wt. % to about 20 wt. %.

In one embodiment, the upgrading solution comprises a polar organic solvent in an amount of greater than or equal to about 10 wt. %. In another embodiment, the upgrading solution comprises polar organic solvent in an amount of about 10 wt. % to about 90 wt. %. In another embodiment, the upgrading solution comprises polar organic solvent in an amount of about 10 wt. % to about 80 wt. %. In another embodiment, the upgrading solution comprises polar organic solvent in an amount of about 10 wt. % to about 70 wt. %. In another embodiment, the upgrading solution comprises polar organic solvent in an amount of about 10 wt. % to about 60 wt. %. In another embodiment, the upgrading solution comprises polar organic solvent in an amount of about 10 wt. % to about 50 wt. %. In another embodiment, the upgrading solution comprises polar organic solvent in an amount of about 10 wt. % to about 40 wt. %. In another embodiment, the upgrading solution comprises polar organic solvent in an amount of about 10 wt. % to about 30 wt. %. In another embodiment, the upgrading solution comprises polar organic solvent in an amount of about 10 wt. % to about 20 wt. %.

In another embodiment, the upgrading solution comprises a pyrolysis oil in an amount of between about 1 to about 50 wt. %, and a polar organic solvent in an amount of about 99 to about 50 wt. %.

In another embodiment, the upgrading solution comprises a pyrolysis oil in an amount of between about 1 to about 50 wt. %, and a polar organic solvent in an amount of about 99 to about 50 wt. %, and water in an amount of 0 to about 10 wt. %.

In another embodiment, the upgrading solution comprises a pyrolysis oil in an amount of between about 10 to about 50 wt. %, and a polar organic solvent in an amount of about 90 to about 50 wt. %.

In another embodiment, the upgrading solution comprises a pyrolysis oil in an amount of between about 10 to about 50 wt. %, and a polar organic solvent in an amount of about 90 to about 50 wt. %, and water in an amount of 0 to about 10 wt. %.

In one embodiment, the upgrading solution comprises pyrolysis oil, a polar organic solvent and optionally water in following proportions:

| Pyrolysis Oil | Polar Organic Solvent | Water |
|---|---|---|
| (wt. %) | (wt. %) | (wt. %) |
| 100% | — | — |
| 50 | 50 | — |
| 50 | 40 | 10 |
| 40 | 50 | 10 |
| 30 | 50 | 20 |
| 10 | 80 | 10 |

In one embodiment, the upgrading solution comprises pyrolysis oil in an amount of about 10 wt. %.

In one embodiment, the upgrading solution comprises pyrolysis oil in an amount of about 10 wt. % and a polar organic solvent in an amount of about 80%.

In one embodiment, the upgrading solution comprises pyrolysis oil in an amount of about 10 wt. %, a polar organic solvent in an amount of about 80% and water in amount of about 10 wt. %.

In one aspect, the present invention relates to the use of an upgrading solution for increasing the concentration of oxygenates in a petroleum product, wherein the upgrading solution comprises a pyrolysis bio-oil and optionally a polar organic solvent.

In one embodiment, the upgrading solution may be as defined in any of the embodiments above.

In one embodiment, the concentration of oxygenates are increased relative to the starting petroleum product. In one embodiment, the starting petroleum product is as defined above. In another embodiment, the starting petroleum product is FCC gasoline.

In one embodiment, the concentration of oxygenates (wt. %) is increased by at least about 40% relative to the concentration of oxygenates (wt. %) in the starting petroleum product.

In one embodiment, the concentration of oxygenates (wt. %) is increased by about 40% to about 500% relative to the concentration of oxygenates (wt. %) in the starting petroleum product. In another embodiment, the concentration of oxygenates (wt. %) is increased by about 40% to about 300% relative to the concentration of oxygenates (wt. %) in the starting petroleum product. In another embodiment, the concentration of oxygenates (wt. %) is increased by about 40% to about 200% relative to the concentration of oxygenates (wt. %) in the starting petroleum product. In another embodiment, the concentration of oxygenates (wt. %) is increased by about 40% to about 150% relative to the concentration of oxygenates (wt. %) in the starting petroleum product.

In another aspect, the present invention relates to the use of an upgrading solution for increasing the octane number of a petroleum product, wherein the petroleum product is a gasoline and wherein the upgrading solution comprises a pyrolysis bio-oil and optionally a polar organic solvent.

The concept of octane numbers is known to the skilled person. The octane number of a liquid hydrocarbon is the measure of the ignition quality when burnt in a standard (spark-ignition internal combustion) engine. The higher this number, the less susceptible the hydrocarbon is to “knocking’ (explosion caused by its premature burning in the combustion chamber). The octane number represents a mixture of isooctane and n-heptane having the same antiknock properties as the fuel, i.e., a hydrocarbon having an octane number of 92 has the same knock as a mixture of 92% isooctane and 8% n-heptane. The octane number can be determined by methods known in the art. The octane number is often determined by means of a standardized test, such as ASTM 2699. In one embodiment, the octane number is determined by standardised test ASTM 2699 (incorporated herein by reference).

In one embodiment, the octane number is increased relative to the starting petroleum product. In one embodiment, the starting petroleum product is as defined above. In another embodiment, the starting petroleum product is FCC gasoline.

In one embodiment, the octane number is increased by about 0.1 to about 50 units relative to the octane number of the starting petroleum product. In another embodiment, the octane number is increased by about 0.1 to about 30 units relative to the octane number of the starting petroleum product. In another embodiment, the octane number is increased by about 0.1 to about 20 units relative to the octane number of the starting petroleum product. In another embodiment, the octane number is increased by about 0.1 to about 5 units relative to the octane number of the starting petroleum product.

In one embodiment, the octane number is increased by about 1 to about 50 units relative to the octane number of the starting petroleum product. In another embodiment, the octane number is increased by about 1 to about 30 units relative to the octane number of the starting petroleum product. In another embodiment, the octane number is increased by about 1 to about 20 units relative to the octane number of the starting petroleum product. In another embodiment, the octane number is increased by about 1 to about 5 units relative to the octane number of the starting petroleum product.

The invention will now be further described by the following numbered paragraphs:
1. A process for upgrading a petroleum product comprising mixing the petroleum product with an upgrading solution to provide a two-phase mixture consisting of an extract phase and a raffinate phase, wherein the upgrading solution comprises a pyrolysis oil and optionally a polar organic solvent.
2. A process according to paragraph 1 wherein the petroleum product and the upgrading solution are intimately mixed.
3. A process according to any one of paragraphs 1 and 2 wherein the petroleum product and the upgrading solution are mixed by stirring, shaking or agitation.
4. A process according to any one of paragraphs 1 and 2 wherein the petroleum product and the upgrading solution are mixed by flow means, such as counter-current flow.
5. A process according to any preceding paragraph wherein the process further comprises separating from the raffinate phase from the extract phase to give an upgraded petroleum product.
6. A process accrording to paragraph 5 wherein the raffinate is separated by draining off the raffinate phase.
7. A process according to any preceding paragraph where the process further comprises mixing the raffinate phase with a further batch of upgrading solution to provide a two-phase mixture consisting of an extract phase and a raffinate phase, wherein the upgrading solution comprises a pyrolysis oil and optionally a polar organic solvent.
8. A process according to any preceding paragraph wherein the mixing is carried out at ambient temperature and pressure.
9. A process according to any preceding paragraph where the raffinate phase has a reduced concentration of sulphur compounds relative to the petroleum product.
10. A process according to paragraph 9 wherein the concentration of sulphur compounds is reduced by about 1% to about 80%, for example, about 5% to about 30% or about 5% to about 20% or about 10% to about 20%, or about 20%.
11. A process according to any preceding paragraph where the raffinate phase has a reduced concentration of olefins relative to the petroleum product.
12. A process according to paragraph 11 where the concentration of olefins is reduced between about 1% and about 80%, for example, between about 5% and about 10%.
13. A process according to any preceding paragraph where the raffinate phase has an increased concentration of oxygenates relative to the petroleum product.
14. A process according to paragraph 13 where the concentration of oxygenates is increase by about 40% to about 500%, for example about 100% to about 200%.
15. A process according to any preceding paragraph where the ratio of petroleum product to upgrading solution is from about 10:1 to about 1:10.
16. A process according to any preceding paragraph where the ratio of the petroleum product to upgrading solution is from about 5:1 to 1:1.
17. A raffinate phase obtainable by a process according to any of the preceding paragraphs.
18. A raffinate phase obtained by a process according to any of the preceding paragraphs.
19. A process for producing an upgraded petroleum product comprising

(i) treating a petroleum product to be upgraded with an upgrading solution,

(ii) mixing the petroleum product and upgrading solution and then allowing the mixture to form two phases consisting of a raffinate phase and an extract phase, and

(iii) separating the raffinate phase from the extract phase to yield an upgraded petroleum product;

wherein the upgrading solution comprises a pyrolysis oil and optionally a polar organic solvent.
20. A process according to paragraph 19 wherein the petroleum product and the upgrading solution are intimately mixed.
21. A process according to any one of paragraphs 19 and 20 wherein the petroleum product and the upgrading solution are mixed by stirring, shaking or agitation.
22. A process according to any one of paragraphs 19 and 20 wherein the petroleum product and the upgrading solution are mixed by flow means, such as counter-current flow.
23. A process according to any one of paragraphs 19 to 22 wherein the raffinate is separated by draining off the raffinate phase.
24. A process according to one of paragraphs 19 to 23 where the process further comprises (iv) mixing the upgraded petroleum product with a further batch of upgrading solution and repeating steps (ii) and (iii), wherein the upgrading solution comprises a pyrolysis oil and optionally a polar organic solvent.
25. A process according to any one of paragraphs 19 to 24 wherein the mixing is carried out at ambient temperature and pressure.
26. A process according to any one of paragraphs 19 to 25 where the upgraded petroleum product has a reduced concentration of sulphur compounds relative to the petroleum product to be upgraded.
27. A process according to paragraph 26 wherein the concentration of sulphur compounds is reduced by about 5% to about 50%, for example, about 5% to about 30% or about 5% to about 20% or about 10% to about 20%, or about 20%.
28. A process according to any one of paragraphs 19 to 27 where the upgraded petroleum product has a reduced concentration of olefins relative to the petroleum product to be upgraded.
29. A process according to paragraph 28 where the concentration of olefins is reduced between about 5% and about 20%, for example, between about 5% and about 10%.
30. A process according to any one of paragraphs 19 to 29 where the upgraded petroleum product has an increased concentration of oxygenates relative to the petroleum product to be upgraded.
31. A process according to paragraph 30 where the concentration of oxygenates is increase by about 40% to about 200%, for example about 100% to about 200%.
32. A process according to any one of paragraphs 19 to 31 where the ratio of petroleum product to be upgraded to upgrading solution is from about 10:1 to about 1:10.
33. A process according to any one of paragraphs 19 to 32 where the ratio of the petroleum product to be upgraded to upgrading solution is from about 5:1 to 1:1.
34. An upgraded petroleum product obtainable by a process according to any one of paragraphs 13 to 27.
35. An upgraded petroleum product obtained by a process according to any one of paragraphs 13 to 27.
36. A process according to any one of paragraphs 1 to 16 and 19 to 33 where the petroleum product to be upgraded is gasoline or diesel.
37. A process according to any one of paragraphs 1 to 16, 19 to 33 and 36 where the petroleum product to be upgraded FCC gasoline.
38. A process according to any one of paragraphs 1 to 16, 19 to 33 and 36 to 37 where the upgrading solution essentially consists of a pyrolysis oil.
39. A process according to any one of paragraphs 1 to 16, 19 to 33 and 36 to 37 where the upgrading solution consists of a pyrolysis oil.
40. A process according to any one of paragraphs 1 to 16, 19 to 33 and 36 to 39 where the pyrolysis oil is obtained/obtainable from fossil fuels, plastic, rubber and biomass.
41. A process according to any one of paragraphs 1 to 16, 19 to 33 and 36 to 40 where the pyrolysis oil is obtained/obtainable from biomass.
42. A process according to paragraph 41 wherein the biomass is selected from wood, crops, agricultural residues and domestic and industrial waste and mixtures thereof.
43. A process according to any one of paragraphs 1 to 16, 19 to 33 and 36 to 42 where the pyrolysis oil is pyrolysis bio-oil.
44. A process according to any one of paragraphs 1 to 16, 19 to 33 and 36 to 43 where the upgrading solution comprises a pyrolysis oil and a polar organic solvent.
45. A process according to any one of paragraphs 1 to 16, 19 to 33 and 36 to 43 where the upgrading solution essentially consists of/consists of a pyrolysis oil and a polar organic solvent.
46. A process according to any one of paragraphs 1 to 16, 19 to 33 and 36 to 45 where the polar organic solvent is an alcohol.
47. A process according to any one of paragraphs 1 to 16, 19 to 33 and 36 to 46 where the polar organic solvent is an alcohol is selected from methanol, ethanol, n-propanol, i-propanol, n-butanol, s-butanol, i-butanol and t-butanol.
48. A process according to any one of paragraphs 1 to 16, 19 to 33 and 36 to 47 where the polar organic solvent is an alcohol is selected from methanol and ethanol.
49. A process according to any one of paragraphs 1 to 16, 19 to 33 and 36 to 48 where the polar organic solvent is methanol.
50. A process according to any one of paragraphs 1 to 16, 19 to 33 and 36 to 49 where the upgrading solution further comprises a solvent selected from water, an alcohol, an aldehyde, a ketone, an ether, a carboxylic acid, an ester, a carbonate, an acid anhydride, an amide, an amine, a heterocyclic compound, an imine, an imide, a nitrile, a nitro compound, a sulfoxide, and a haloalkane.
51. A process according to any one of paragraphs 1 to 16, 19 to 33 and 36 to 50 where the upgrading solution further comprises a solvent selected from water, an alcohol, a ketone, an ether, an ester, and a nitrile.
52. A process according to any one of paragraphs 1 to 16, 19 to 33 and 36 to 51 where the upgrading solution further comprises a solvent selected from water and an alcohol.
53. A process according to any one of paragraphs 1 to 16, 19 to 33 and 36 to 52 where the upgrading solution further comprises a solvent selected from water, ethylene glycol, propylene glycol, and propane-1,3-diol.
54. A process according to any one of paragraphs 1 to 16, 19 to 33, 36 and 37 where the upgrading solution comprises (i) a pyrolysis bio-oil, (ii) a polar organic solvent selected from methanol, ethanol, propanol, isopropanol, butanol, sec-butanol, iso-butanol, (iii) at least one further alcohol solvent selected from ethylene glycol, propylene glycol, and propane-1,3-diol; and (iv) water.
55. A process according to any one of paragraphs 1 to 16, 19 to 33, 36 and 37 where the upgrading solution comprises (i) a pyrolysis bio-oil, (ii) a polar organic solvent selected from methanol, ethanol, propanol, isopropanol, butanol, sec-butanol, iso-butanol, (iii) ethylene glycol; and (iv) water.
56. A process according to any one of paragraphs 1 to 16, 19 to 33, 36 and 37 where the upgrading solution comprises (i) a pyrolysis bio-oil, (ii) a polar organic solvent selected from methanol, ethanol, propanol, isopropanol, butanol, sec-butanol, iso-butanol, and (iii) water.
57. A process according to any one of paragraphs 1 to 16, 19 to 33, 36 and 37 where the upgrading solution comprises (i) a pyrolysis bio-oil, (ii) a polar organic solvent selected from methanol and ethanol and (iii) water.
58. A process according to any one of paragraphs 1 to 16, 19 to 33, 36 and 37 where the upgrading solution comprises (i) a pyrolysis bio-oil, and (ii) a polar organic solvent selected from methanol and ethanol.
59. A process according to any one of paragraphs 1 to 16, 19 to 33 and 36 to 58 where the upgrading solution comprises the pyrolysis oil in an amount of greater than or equal to about 10 wt. %, suitably in amount of about 10 wt. % to about 90 wt. %, suitably about 10 wt. % to about 60 wt. %.
60. A process according to any one of paragraphs 1 to 16, 19 to 33 and 36 to 59 where the upgrading solution comprises the polar organic solvent in an amount of greater than or equal to about 10 wt. %, suitably in amount of about 10 wt. % to about 90 wt. %, suitably about 10 wt. % to about 60 wt. %.
61. A process according to any one of paragraphs 1 to 16, 19 to 33 and 36 to 59 where the upgrading solution comprises pyrolysis oil in an amount of about 10 wt. % and a polar organic solvent in an amount of about 80%.
62. A process according to any one of paragraphs 1 to 16, 19 to 33 and 36 to 59 where the upgrading solution comprises pyrolysis oil in an amount of about 10 wt. %, a polar organic solvent in an amount of about 80%, and water in an amount of about 10 wt. %.
63. An upgrading solution for upgrading petroleum products comprising a pyrolysis oil and optionally a polar organic solvent.
64. An upgrading solution according to paragraph 63 where the upgrading solution consists of a pyrolysis oil.
65. An upgrading solution according to any one of paragraphs 63 and 64 where the pyrolysis oil is obtained/obtainable from fossil fuels, plastic, rubber and biomass.
66. An upgrading solution according to any one of paragraphs 63 to 65 where the pyrolysis oil is obtained/obtainable from biomass.
67. An upgrading solution according to paragraph 66 where the biomass is selected from wood, crops, agricultural residues and domestic and industrial waste and mixtures thereof.
68. An upgrading solution according to any one of paragraphs 63 to 67 where the pyrolysis oil is pyrolysis bio-oil.
69. An upgrading solution according to any one of paragraphs 63 to 68 where the upgrading solution comprises a pyrolysis oil and a polar organic solvent.
70. An upgrading solution according to any one of paragraphs 63 to 69 where the upgrading solution essentially consists of a pyrolysis oil and a polar organic solvent.
71. An upgrading solution according to any one of paragraphs 63 to 70 where the upgrading solution consists of a pyrolysis oil and a polar organic solvent.
72. An upgrading solution according to any one of paragraphs 63 to 71 where the polar organic solvent is an alcohol.
73. An upgrading solution according to any one of paragraphs 63 to 72 where the polar organic solvent is an alcohol is selected from methanol, ethanol, n-propanol, i-propanol, n-butanol, s-butanol, i-butanol and t-butanol.
74. An upgrading solution according to any one of paragraphs 63 to 73 where the polar organic solvent is an alcohol is selected from methanol and ethanol.
75. An upgrading solution according to any one of paragraphs 63 to 74 where the polar organic solvent is methanol.
76. An upgrading solution according to any one of paragraphs 63 to 75 where the upgrading solution further comprises a solvent selected from water, an alcohol, an aldehyde, a ketone, an ether, a carboxylic acid, an ester, a carbonate, an acid anhydride, an amide, an amine, a heterocyclic compound, an imine, an imide, a nitrile, a nitro compound, a sulfoxide, and a haloalkane.
77. An upgrading solution according to any one of paragraphs 63 to 76 where the upgrading solution further comprises a solvent selected from water, an alcohol, a ketone, an ether, an ester, and a nitrile.
78. An upgrading solution according to any one of paragraphs 63 to 77 where the upgrading solution further comprises a solvent selected from water and an alcohol.
79. An upgrading solution according to any one of paragraphs 63 to 78 where the upgrading solution further comprises a solvent selected from water, ethylene glycol, propylene glycol, and propane-1,3-diol.
80. An upgrading solution according to any one of paragraphs 63 to 79 where the upgrading solution comprises (i) a pyrolysis bio-oil, (ii) a polar organic solvent selected from methanol, ethanol, propanol, isopropanol, butanol, sec-butanol, iso-butanol, (iii) at least one further alcohol solvent selected from ethylene glycol, propylene glycol, and propane-1,3-diol; and (iv) water.
81. An upgrading solution according to any one of paragraphs 63 to 79 where the upgrading solution comprises (i) a pyrolysis bio-oil, (ii) a polar organic solvent selected from methanol, ethanol, propanol, isopropanol, butanol, sec-butanol, iso-butanol, (iii) ethylene glycol; and (iv) water.
82. An upgrading solution according to any one of paragraphs 63 to 79 where the upgrading solution comprises (i) a pyrolysis bio-oil, (ii) a polar organic solvent selected from methanol, ethanol, propanol, isopropanol, butanol, sec-butanol, iso-butanol, and (iii) water.
83. An upgrading solution according to any one of paragraphs 63 to 79 where the upgrading solution comprises (i) a pyrolysis bio-oil, (ii) a polar organic solvent selected from methanol and ethanol and (iii) water.
84. An upgrading solution according to any one of paragraphs 63 to 79 where the upgrading solution comprises (i) a pyrolysis bio-oil, and (ii) a polar organic solvent selected from methanol and ethanol.
85. An upgrading solution according to any one of paragraphs 63 to 79 where the upgrading solution comprises the pyrolysis oil in an amount of greater than or equal to about 10 wt. %, suitably in amount of about 10 wt. % to about 90 wt. %, suitably about 10 wt. % to about 60 wt. %.
86. An upgrading solution according to any one of paragraphs 63 to 79 where the upgrading solution comprises the polar organic solvent in an amount of greater than or equal to about 10 wt. %, suitably in amount of about 10 wt. % to about 90 wt. %, suitably about 10 wt. % to about 60 wt. %.
87. An upgrading solution according to any one of paragraphs 63 to 79 where the upgrading solution comprises pyrolysis oil in an amount of about 10 wt. % and a polar organic solvent in an amount of about 80%.
88. An upgrading solution according to any one of paragraphs 63 to 79 where the upgrading solution comprises pyrolysis oil in an amount of about 10 wt. %, a polar organic solvent in an amount of about 80%, and water in an amount of about 10 wt. %.
89. Use of an upgrading solution for increasing the concentration of oxygenates in a petroleum product, wherein the upgrading solution comprises a pyrolysis bio-oil and optionally a polar organic solvent.
90. Use according to paragraph 89 wherein the upgrading solution is as defined in any one of paragraphs 63 to 89.
91. Use according to any one of paragraphs 89 and 90 wherein the concentration of oxygenates (wt. %) is increased by about 40% to about 200% relative to the initial concentration of oxygenates (wt. %) in the petroleum product.
92. Use of an upgrading solution for increasing the octane number of a petroleum product, wherein the upgrading solution comprises a pyrolysis bio-oil and optionally a polar organic solvent.
93. Use according to paragraph 92 wherein the upgrading solution is as defined in any one of paragraphs 63 to 89.
94. Use according to any one of paragraphs 92 and 93 wherein the octane number is increased by about 1 to about 10 units relative to the octane number of the initial petroleum product.
95. Use according to any one of paragraphs 89 to 94 wherein the petroleum product is gasoline, suitably FCC gasoline.

### EXAMPLES

### Materials

Methanol with purity of 99.9% was purchased from Sigma Aldrich Company. Pyrolysis (Bio)-oil was purchased from Future Blends Limited, UK. FCC gasoline (unpurified) was purchased from Petroineos Manufacturing Scotland LTD, UK. These agents were used without further purification.

### Upgrading Solution

An upgrading solution was prepared by mixing methanol (80%, wt), Bio-oil (10%, wt) and distilled water (10%, wt).

### Upgrading Process

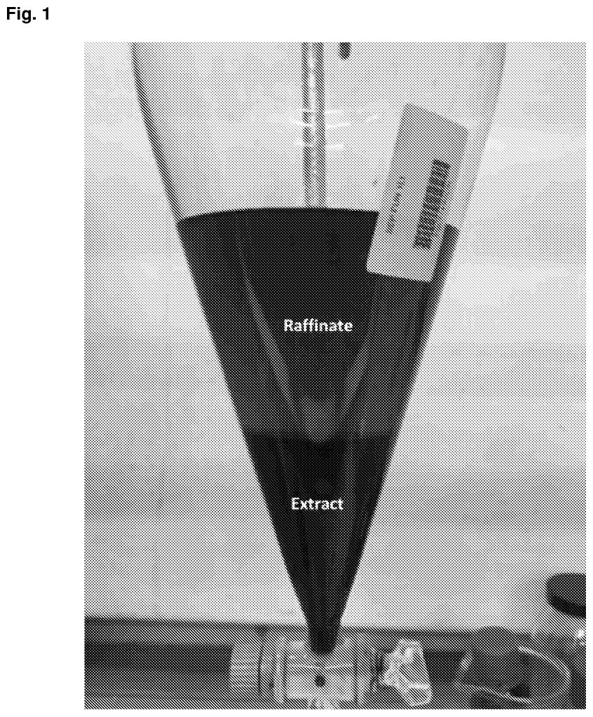

An upgrading solution was prepared as described above. FCC gasoline and upgrading solution were fed into a separating funnel in a ratio (wt. %) of 5:1 FCC gasoline to upgrading solution. The two phases were mixed well by vigorously shaking the separating funnel. The mixture was allowed to stand and two liquid phases were observed in the mixture, the mixture was stabilized for another 5 minutes (FIG. 1). The upper phase (raffinate) and lower phase (extract) were separated, and fresh upgrading solution was added into the upper phase (raffinate) with 5:1 ratio (wt. %) raffinate phase to upgrading solution. This process was repeated another 4 times and the raffinate phase was isolated.

### Analysis

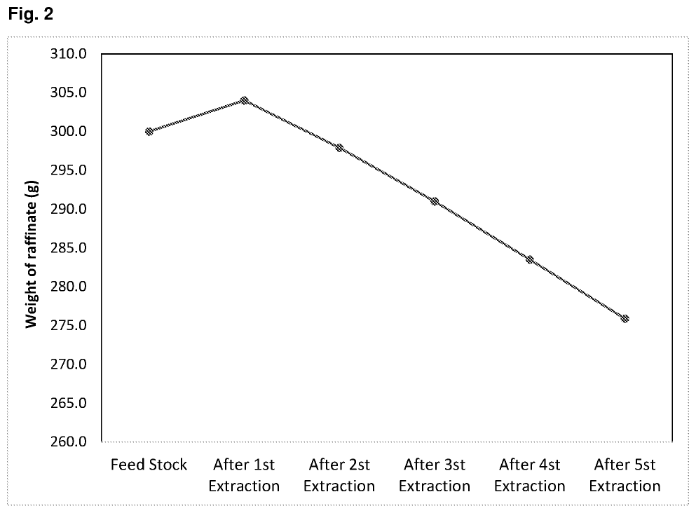

The weight of the raffinate was measured after each extraction (FIG. 2). After the first extraction a weight increase was observed in raffinate phase (relative to starting FCC gasoline). The raffinate phase weight then slightly decreased with each extraction with fresh upgrading solution (2^{nd} to 5^{th}). After the last extraction (5^{th}), a relative weight loss of 8.03% had occurred.

Following the upgrading process, Gas Chromatography-Mass Spectrometry (GCMS) was employed to quantify the level of various components in the raffinate phase. For comparison, the starting FCC gasoline was also analysed by the GCMS.

The GCMS analyser was a SHIMADZU GCMS-QP2010 SE gas chromatography mass spectrometer. The main operating parameters of GCMS analyser were: Column Oven Temperature 35° C./308K; Injection Temperature 205° C./478 K; Injection Mode direct; Temperature rising rate from 35 to 40° C. was 1° C./min, and after 40° C. the rate was changed to 10° C./min, until it reached 180° C./453 K.

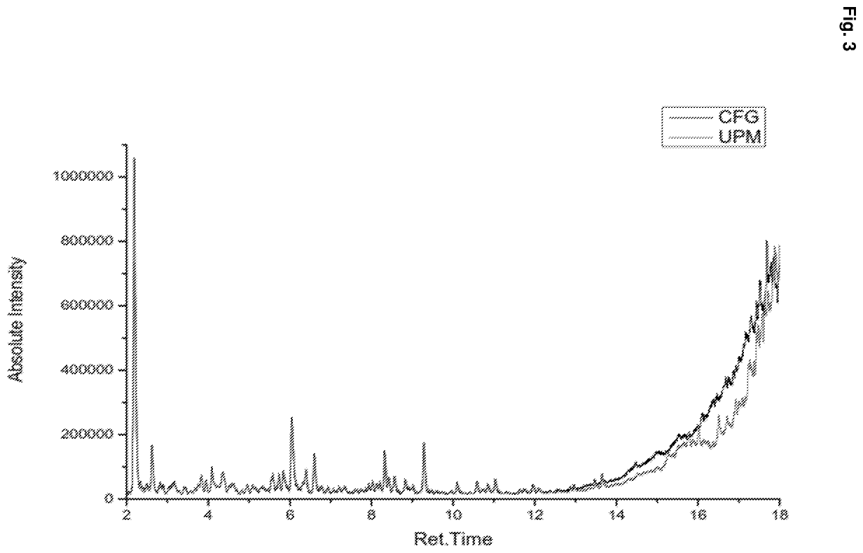

A comparison of the GCMS results of starting FCC gasoline and the raffinate showed that the raffinate had a decreased proportion of C₅-C₉+ olefins, and aromatics and an increased proportion of oxygenates (FIG. 3). SI-PIONA analysis (Thermo Fischer Scientific Trace 1300GC) provided the following component report (Table 1).

TABLE 1
Component Report

| | Sulphur content | Olefins | Aromatics |
|---|---|---|---|
| | (ppm) | v % | v % |
| Starting FCC | 959.9 | 30.39 | 31.80 |
| gasoline | | | |
| Raffinate phase | 768.5 | 25.39 | 30.80 |

Integration of the area under the oxygenates peak in the GCMS of the raffinate phase revealed the distribution of oxygenates present (Table 2). It was found that 3.5% (% Area in GCMS) of oxy-organics (oxygenates) in total were transferred from the upgrading solution to the raffinate phase.

TABLE 2
Distribution of oxy-organics (oxygenates) contents in raffinate

| | |
|---|---|
| Starting FCC | Raffinate |
| gasoline | phase |

| | |
|---|---|
| Type of oxygenate | % Area (GCMS) |

| | | |
|---|---|---|
| Alcohols (including methanol) | 0.82 | 1.75 |
| Nitrogen-containing oxygenates | 0.88 | 1.23 |
| Carboxylic acid and derivatives, etc | | 0.27 |
| Aldehydes | | 0.14 |
| Ketones | 0.04 | 0.11 |
| Total | 1.74 | 3.5 |

The stability of the raffinate was assessed by storing it for one month at ambient temperature and pressure in a sealed amber glass bottle. The raffinate was then reanalysed by GCMS.

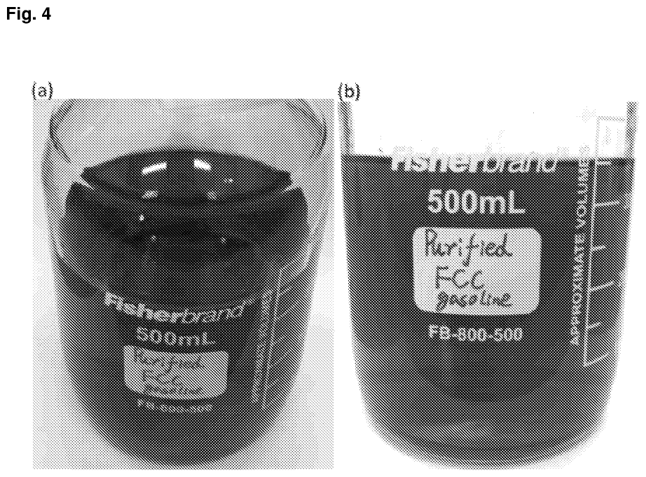

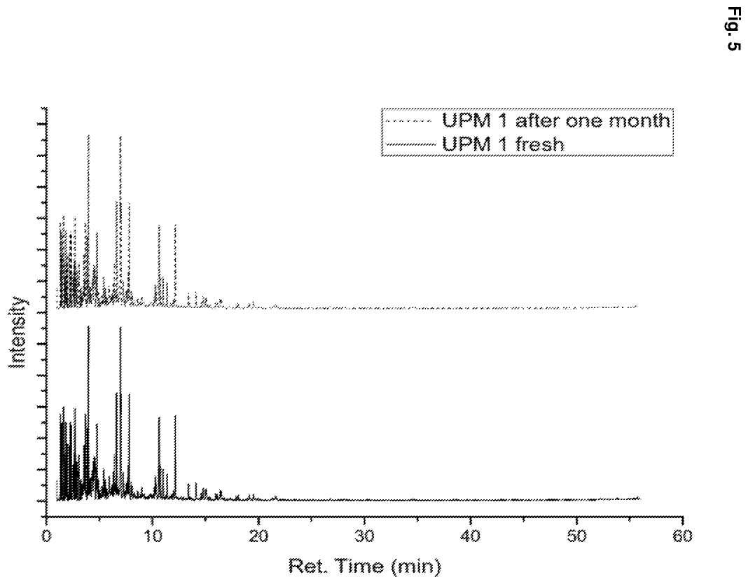

It was found that there was no obvious colour change after one month storage (FIG. 4). On the other hand, GCMS analysis (FIG. 5) results also indicated that there is no obvious change in the composition of the raffinate phase/upgraded petroleum product.

Gasoline is formed by a complex mixture of paraffins, isoparaffins, olefins, naphthenics, aromatics and some amount of other compounds, with chains containing 4 to 12 atoms of carbon, and with a range of ebullition of from 30 to 225° C. In order to prove the raffinate phase from the upgrading process of the present example can be considered as a gasoline product, the boiling point under atmosphere was analysed. The analyser used was a TA Instruments SDT Analyzer Model Q600. The analysis program was: 100 ml/min carrier gas flow rate (N₂), 5° C./min heating rate, final temperature is 600° C.

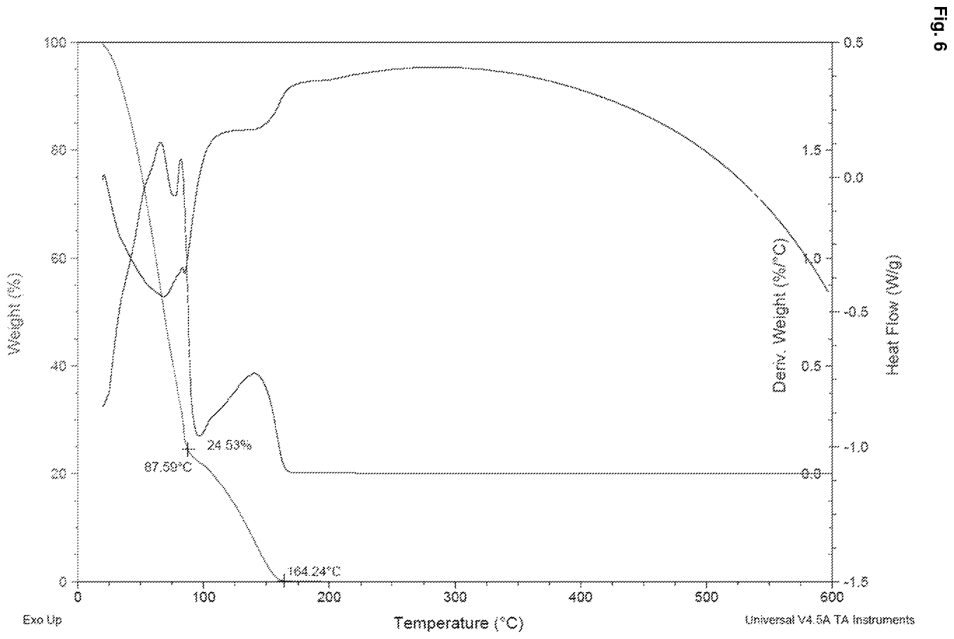

From the TGA analysis result of the raffinate, it was confirmed that all the components in the raffinate evaporated before the temperature of the furnace reached 165° C. (FIG. 6). It is noted that the raffinate phase consists of a two part of mixture with different ebullition temperatures. Firstly, the major part (75.47% wt) of the raffinate evaporated before 88° C., and secondly, the rest of the raffinate evaporated between 88 and 165° C. (FIG. 6).

Table 3 below provide further analysis of (A) the starting FCC gasoline, (B) the raffinate produced using an upgrading solution consisting of 90 wt % MeOH and 10 wt % water; and also (C) the raffinate produced using an upgrading solution consisting of 80 wt % MeOH/10 wt % water/10 wt % pyrolysis bio-oil.

TABLE 3
Sulphur, hydrocarbon and octane analysis

| | A | B | C |
|---|---|---|---|
| Sulphur (ppm) | 959.9 | 777.9 | 768.5 |
| Paraffin, v % | 4.23 | 4.42 | 4.4 |
| Iso-paraffin, v % | 20.31 | 22.76 | 22.66 |
| Olefins, v % | 30.39 | 25.37 | 25.39 |
| Naphthenes, v % | 13.27 | 16.71 | 16.75 |
| Aromatics, v % | 31.8 | 30.74 | 30.8 |
| RON (not considered | 89.39 | 88.96 | 89 |
| oxygenates) | | | |
| MON (not considered | 80.59 | 80.03 | 80.02 |
| oxygenates) | | | |

Further upgrading solutions were prepared using the method described above. The compositions of these upgrading solutions are provided in Table 4 below

TABLE 4
Composition of the upgrading solutions

| | Pyrolysis | Polar Organic | |
|---|---|---|---|
| Upgrading | Bio-Oil | Solvent | Water |
| Solution | (wt. %) | (wt. %) | (wt. %) |
| 2 | 30 | 60 | 10 |
| 3 | 50 | 40 | 10 |
| 4 | 50 | 50 | — |
| 5 | 100 | — | — |

Integration of the area under the oxygenates peak in the GCMS of the raffinate phases produced when using upgrading solutions 2 to 5 in the process of the invention revealed the distribution of oxygenates present (Table 5).

TABLE 5
Distribution of oxy-organics (oxygenates)
contents in FCC gasoline and raffinates

| Type of | FCC | | | | |
|---|---|---|---|---|---|
| oxy-organics | Gasoline | 2 | 3 | 4 | 5 |
| (oxygenates) | % Area | % Area | % Area | % Area | % Area |
| Alcohols (without | 0.82 | 1.12 | 1.06 | 1.25 | 1.24 |
| methanol) | | | | | |
| Nitrogen-containing | 0.88 | 0.52 | 0.54 | 0.56 | 0.58 |
| oxygenates | | | | | |
| Carboxylic acid | | 0.46 | 0.48 | 0.46 | 0.48 |
| and derivatives | | | | | |
| Aldehydes | | 0.27 | 0.27 | 0.28 | 0.28 |
| Ketones | 0.04 | 0.26 | 0.27 | 0.31 | 0.35 |
| Phenols | | 0.11 | 0.18 | 0.13 | 0.84 |
| Total | 1.74 | 2.74 | 2.8 | 2.99 | 3.77 |

The total oxygenates contents (% Area in GCMS, without methanol) increased with the concentration of the bio-oil in the blending mixtures (Table 5). With 100% bio-oil blending mixture the total contents of oxygenates in blended gasoline (UPM) can achieve 3.77% (% Area in GCMS).

### Conclusion

An upgrading solution comprising a pyrolysis-oil based was used to upgrade commercial FCC gasoline. The upgrading solution showed excellent olefin reduction and desulfurization performance. Furthermore, the upgrading solution improved the oxygenate content of the FCC gasoline and increased the octane number.

The upgraded petroleum product was found to be storage stable.

The process of the present invention provides a quick and efficient process for upgrading FCC gasoline without the need for the supply of external hydrogen and harsh reaction conditions.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise paragraphed. No language in the specification should be construed as indicating any non-paragraphed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the paragraphs appended hereto as permitted by applicable law.

1. A process for upgrading a petroleum product comprising mixing the petroleum product with an upgrading solution to provide a two-phase mixture consisting of an extract phase and a raffinate phase, wherein the upgrading solution comprises a pyrolysis oil and optionally a polar organic solvent.

2. The process of claim 1, further comprising
separating the raffinate phase from the extract phase to yield an upgraded petroleum product.

3. The process of claim 1, wherein the ratio of petroleum product to upgrading solution is from about 10:1 to about 1:10.

4. The process of claim 1, wherein the petroleum product is FCC gasoline.

5. The process of claim 1, wherein the pyrolysis oil is selected from one or more of a rubber pyrolysis oil, a plastic pyrolysis oil, a fossil fuel pyrolysis oil and a pyrolysis bio-oil.

6. The process according to claim 5 wherein the pyrolysis oil is a pyrolysis bio-oil.

7. The process of claim 1, wherein the upgrading solution comprises a polar organic solvent selected from methanol, ethanol, n-propanol, i-propanol, n-butanol, s-butanol, i-butanol and t-butanol.

8. The process of claim 1, wherein the upgrading solution comprises (i) a pyrolysis bio-oil, (ii) a polar organic solvent selected from methanol, ethanol, propanol, isopropanol, butanol, sec-butanol, iso-butanol, (iii) at least one further alcohol solvent selected from ethylene glycol, propylene glycol, and propane-1,3-diol; and (iv) water.

9. The process of claim 1, wherein the upgrading solution comprises (i) a pyrolysis bio-oil, and (ii) a polar organic solvent selected from methanol and ethanol.

10. The process of claim 1, wherein the upgrading solution comprises the pyrolysis oil in an amount of greater than or equal to about 10 wt. %.

11. An upgrading solution for upgrading petroleum products comprising a pyrolysis oil and optionally a polar organic solvent.

12. The upgrading solution of claim 11, wherein the pyrolysis oil is selected from one or more of a rubber pyrolysis oil, a plastic pyrolysis oil, a fossil fuel pyrolysis oil and pyrolysis bio-oil.

13. The upgrading solution of claim 12 wherein the pyrolysis oil is a pyrolysis bio-oil.

14. The upgrading solution of claim 11, wherein the upgrading solution comprises pyrolysis oil in amount of greater than or equal to about 10 wt. %.

15. A method of increasing the concentration of oxygenates in a petroleum product, comprising mixing the petroleum product with an upgrading solution to provide a two-phase mixture consisting of an extract phase and a raffinate phase, wherein the upgrading solution comprises a pyrolysis bio-oil and optionally a polar organic solvent.

16. A method of increasing the octane number of a petroleum product, comprising mixing the petroleum product with an upgrading solution to provide a two-phase mixture consisting of an extract phase and a raffinate phase, wherein the petroleum product is gasoline, and wherein the upgrading solution comprises a pyrolysis bio-oil and optionally a polar organic solvent.

17. An upgraded petroleum product obtainable by a process according to claim 1.

18. The upgrading solution of claim 11, wherein the polar organic solvent is selected from methanol, ethanol, n-propanol, i-propanol, n-butanol, s-butanol, i-butanol and t-butanol.

19. The upgrading solution of claim 11, wherein the pyrolysis oil is a pyrolysis bio-oil, the polar organic solvent is selected from methanol, ethanol, propanol, isopropanol, butanol, sec-butanol, iso-butanol, and further comprising at least one further alcohol solvent selected from ethylene glycol, propylene glycol, and propane-1,3-diol; and (iv) water.

20. The upgrading solution of claim 11, wherein the pyrolysis bio-oil is a pyrolysis bio-oil, and the polar organic solvent is selected from methanol and ethanol.
